(19) United States
(12) Patent Application Publication
(10) Pub. No.: US 2015/0301132 A1
(43) Pub. Date: Oct. 22, 2015

(54) RHEOLOGY UNIT FOR MRI WITH INTEGRATED OSCILLATOR AND RF ANTENNA

(71) Applicant: KONINKLIJKE PHILIPS N.V., Eindhoven (NL)

(72) Inventors: DANIEL WIRTZ, HANBURG (DE); PETER VERNICKEL, HAMBURG (DE); PETER MAZURKEWITZ, HAMBURG (DE); CHRISTOPH LEUSSLER, HAMBURG (DE)

(21) Appl. No.: 14/441,864

(22) PCT Filed: Nov. 4, 2013
(86) PCT No.: PCT/IB2013/059876

§ 371 Date: May 11, 2015

Related U.S. Application Data
(60) Provisional application No. 61/725,065, filed on Nov. 12, 2012.

(51) Int. Cl. G01R 33/34; G01R 33/3415; A61B 5/055

(52) U.S. Cl. CPC G01R 33/34084; A61B 5/055; G01R 33/3415

(57) ABSTRACT

The present invention provides a rheology module (200) for use in a magnetic resonance (MR) rheology imaging system (110), whereby the rheology module (200) is adapted to introduce mechanical oscillations into a subject of interest (120), comprising a housing (202), a mechanical oscillator unit (204), which extends at least partially outside the housing (202) and is movable relative to the housing (202), and a transducer (206) for moving the oscillator unit (204), whereby the rheology module (200) comprises at least one radio frequency (RF) antenna unit (210, 212), which comprises at least one RF coil (214, 216). With the RF antenna device integrated into the rheology module, an antenna placement close to a region of interest (ROI) can be achieved to improve the MR imaging capabilities of a MR rheology imaging system. Thus, imaging of the ROI can be performed more efficiently. Furthermore, connection and cabling can be facilitated, since only one module has to be connected to generate the oscillation and to operate the RF antenna device.

### FIELD OF THE INVENTION

The invention relates to the field of magnetic resonance (MR) rheology imaging. In particular, the invention relates to the field of rheology modules for use in a MR rheology imaging system, whereby the rheology module is adapted to introduce mechanical oscillations into a subject of interest. Furthermore, the invention relates to the field of rheology arrangements for use in a MR rheology imaging system, comprising at least one RF antenna module comprising at least one RF antenna unit, and at least one rheology module as specified above. Still further, the invention relates to the field of MR rheology imaging systems comprising a rheology module as specified above.

### BACKGROUND OF THE INVENTION

In the area of magnetic resonance (MR) imaging, MR rheology is a technique for gathering additional information on tissue properties, which is not accessible with MR imaging alone. Parameters like tissue viscosity or elasticity can otherwise only be determined using biopsy and/or histology. On the other hand it has been demonstrated, that these tissue properties can help in the detection of cirrhotic or cancerous changes, e.g. in liver, breast or brain tissue. In particular, MR rheology has been proven to be especially useful for determining and staging liver cirrhosis as well as breast cancer. Initial applications to degenerative brain diseases have also been proposed.

Typically a MR rheology setup contains a rheology module for introducing mechanical oscillations into a subject of interest and an MR imaging system. In order to determine tissue properties of a particular region of interest (ROI) of the subject of interest, the rheology module is placed close to this area to introduce the oscillations. Appropriate radio frequency (RF) antenna units comprising one or more RF coils are provided for generating the anatomical ‘background’ images. These RF antenna units can be part of the MR imaging system, or additional RF antenna units can be provided in RF antenna modules to improve the imaging in respect to the ROI of the subject of interest. Particularly, the RF antenna modules are provided close to the ROI.

For some MR measurements, a rheology arrangement is used. The arrangement comprises a rheology module and typically at least one RF antenna module comprising at least one RF antenna unit. These modules are combined and interconnected, so that the rheology arrangement can be placed at the subject of interest or close to the subject of interest. In general, the closer a RF antenna unit is located to the ROI, the better are the results of the MR rheology imaging process. Due to the size of the modules, placement of the RF antenna units is usually not as close to the ROI as desired. The optimum size of the RF-coil to the ROI is determined by this distance. Thus, large coils can image with good results even from a larger distance. A field-of-view (FOV) of such coils on the other hand is relatively large and may not be appropriate for the MR rheology imaging process. If the ROI is rather small and/or close to the surface of the subject of interest, small receive coils perform better due to reduced noise pickup from the surroundings outside the ROI. Moreover a larger number of coil elements allows for speeding up the imaging process using algorithms like SENSE. With few coil elements such an acceleration in imaging is not possible.

Furthermore, cabling of the rheology module and the RF antenna modules is rather complicated. Apart from signaling lines, also a supply line is a required for providing power to the rheology module as well as the RF antenna module. Cabling needs increase the setup time for performing MR rheology measurements, and can even reduce the imaging performance of the MR rheology system. Accordingly, improvements are desired.

A magnetic resonance elastography (MRE) scan is known from U.S. Pat. No. 7,307,423 B2. The scan is performed using an array of transducers for applying a strain wave to tissues in a region of interest. A calibration process is performed prior to the scan in which the strain wave produced by each transducer in the array is imaged using an MRE pulse sequence so that information may be acquired that enables each transducer to be properly driven during a subsequent MRE scan.

### SUMMARY OF THE INVENTION

It is an object of the invention to provide a rheology module, a rheology arrangement comprising such a module, and a MR rheology imaging system, which enable improved MR rheology imaging, and which facilitate and improve a workflow for performing MR rheology imaging.

In one aspect of the present invention, the object is achieved by a rheology module for use in a magnetic resonance (MR) rheology imaging system, whereby the rheology module is adapted to introduce mechanical oscillations into a subject of interest, comprising a housing, a mechanical oscillator unit, which extends at least partially outside the housing and is movable relative to the housing, and a transducer for moving the oscillator unit, whereby the rheology module comprises at least one radio frequency (RF) antenna unit, which comprises at least one RF coil.

With the RF antenna device integrated into the rheology module, an antenna placement close to a region of interest (ROI) can be achieved to improve the MR imaging capabilities of a MR rheology imaging system. Thus, imaging of the ROI can be performed more efficiently. Furthermore, connection and cabling can be facilitated, since only one module has to be connected to generate the oscillation and to operate the RF antenna device. The RF antenna device can be used as transmit/receive or receive only device, where each coil can be a receive coil only or a local transmit/receive coil. The MR rheology module overcomes disadvantages of the separate placement of the mechanical oscillator unit and the RF antenna device. Accordingly, also cable routing can be improved and interaction between mechanical oscillator unit and MR imaging equipment can be reduced. The geometry of the module can be freely chosen. Also the geometry of the RF antenna units and RF coils can be chosen as required. Typically, the RF coils have an essentially circular or rectangular shape. Further preferred, the essentially circular shape can be formed by six to eight linear segments, e.g. in the form of a stop sign or octagon. Preferably, the RF antenna unit is provided at the side of the rheology module, where the mechanical oscillator comes into contact with the subject of interest. Any suitable kind of transducer can be used to generate the oscillation of the mechanical oscillator unit, including an electrical, a pneumatic, or a hydraulic transducer. The electrical transducer is preferably a piezoelectric transducer, which converts electric energy into mechanical energy or an electromechanical transducer making use of the movement of a current driven coil within the static magnetic field of the MR imaging device. Piezoelectric transducers can respond very rapidly to drive voltage changes. In a preferred embodiment, the rheology module is provided with a thin housing, which facilitates its placement. The transducer can be provided as a backpack with a local, heavy mass to provide sufficient mechanical inertia to keep amplitude of the oscillations high. The local mass can be exchanged with respect to a desired application, e.g. for different organs or depending on the thickness of fatty tissue layer. The local mass is chosen to be MR compatible.

According to a preferred embodiment at least one RF antenna unit is located at the housing. Different placements of the RF antenna unit at the housing are possible. The RF antenna unit is preferably arranged to surround the oscillator unit. The RF antenna unit can be provided at an upper surface of the housing, integrated into the housing, or at an inner surface thereof. Preferably, the RF antenna unit is provided over an entire face of the housing, e.g. the face facing the subject of interest in operation.

According to a preferred embodiment at least one RF antenna unit is located at the oscillator unit. The RF antenna unit can be provided at an upper surface of the oscillator unit, so that the RF antenna unit is in direct contact with the subject of interest. Alternatively, the RF antenna unit can be integrated into the oscillator unit, or the RF antenna unit can be located at a lower surface thereof facing the housing.

According to a preferred embodiment at least one RF antenna unit comprises a set of multiple RF coils. Multiple RF coils can be used for image generation with increased speed for dynamic processes. The RF coils of the RF antenna unit can be arranged in different ways, e.g. in an array. The RF coils can also be arranged to overlap with adjacent coils and hence being geometrically decoupled from each other. The RF antenna unit can also comprise at least one local, separate transmit coil and a separate receive coil array. For more than one transmit channel, the integrated transmit coil has a nearby RF power combiner to distribute RF power to different transmit coil elements. Accordingly, the MR rheology unit can be used independent from the presence of a large integrated body transmit coil. The receive coil array is detuned during the transmit pulse and the transmit coil is detuned during reception of MRI signals.

According to a preferred embodiment the transducer converts electrical energy into mechanical oscillations, and the rheology module comprises an electrical connector, whereby the electrical connector is provided as a single harness to provide electrical power and a signaling connection to the transducer and the at least one RF antenna unit. With the single harness, connection of the MR rheology module can easily be performed. The harness can comprise individual lines for power and signaling for the transducer and the RF antenna unit. The signaling line can be provided as bi-directional line for sending signals to the transducer and/or the RF antenna unit and receiving signals from the transducer and/or the RF antenna unit. Nevertheless, also independent signaling lines can be provided. Furthermore, multiple signaling lines can be provided for transmitting different kinds of signals. The harness can be further provided with a power line for the transducer and/or the RF antenna unit. A typical rheology module has four types of lines for the RF Antenna device, which are DC feed, RF signal, detune, and malfunction detection, and two lines for the transducer, which are a driving signal and a sensing line to monitor the performance of the transducer. Only one supply line for RF coils and transducer can be realized using a local semiconductor FET switch or amplifier inside the oscillator unit. A local amplifier can directly drive the transducer. Accordingly, B₀-compensation for the power line has to be performed only once.

According to a preferred embodiment the harness comprises at least one line, which is connected to the transducer and the at least one RF antenna unit, and a filter unit provided in the line for splitting signals received from the line according to their frequency, whereby electrical signals on the line are provided from the filter unit to the transducer and the RF antenna unit depending on their frequency. The line can be a signaling line. Preferably, the line is a combined signaling and power line. A typical MR signal has a frequency of some 10 MHz, power supply for the RF antenna device, in particular for preamplifiers associated to the RF antenna device, is a DC signal, and the transducer is driven by a signal having some 10 Hz.

According to a preferred embodiment the harness comprises at least one digital signaling line, the rheology module comprises an AD/DA converter unit, which is connected to the digital signaling line, the transducer and the at least one RF antenna unit, and the AD/DA converter unit is adapted to perform a conversion and allocation of signals between the digital signaling line and the transducer and the at least one RF antenna unit. The signal conversion refers to AD/DA conversion. The allocation of the signals to the transducer and the RF antenna unit refers to multiplexing of signals from different RF coils to be transmitted, or to a separation of received signals, so that they can be provided to the correct recipient, i.e. the corresponding RF coil or the transducer. The digital line can be a bi-directional line, or a unidirectional line. The AD/DA converter is adapted to perform the required conversion. The AD conversion of the RF signal is preferably done on the RF antenna device, further preferred in the RF coil of the RF antenna device.

According to a preferred embodiment the digital signaling line is an optical signaling line. The optical signaling line enables high data transmission rates. Furthermore, the influence on the magnetic fields of the MR rheology imaging system is reduced compared to an electrical line, which generates a magnetic field when electrical signals are transmitted. The optical line is preferably a standard optical high-speed data connection.

According to a preferred embodiment the housing is flexible. The flexible housing facilitates the positioning of the rheology module. Furthermore, the rheology module can adapt to the form of the subject of interest, so that the RF antenna device can be in close contact thereto.

In another aspect of the present invention, the object is achieved by a rheology arrangement for use in a magnetic resonance (MR) rheology imaging system, comprising at least one RF antenna module comprising at least one RF antenna unit, and at least one rheology module as specified above, whereby the at least one RF antenna module and the at least one rheology module are interconnected. The modules can be arranged in any suitable way, e.g. as an array with multiple modules arranged in two directions, or as chain, where the modules are arranged in only one direction. Preferably, also an electrical connection between the modules is provided, so that the rheology arrangement can be easily connected. Further preferred, the rheology arrangement is provided with a single connector for electrically connecting all modules. The modules can be combined, i.e. attached to each other, outside the MR rheology imaging system. Preferably, a reversible connecting method is used for interconnecting the modules, e.g. zip or Velcro fastener. This allows easily adapting the applicator and coil array to the subject of interest and furthermore the ROI. The rheology arrangement can be tailored to fit the desired imaging/rheology measurement depending on the application, thus providing superior images.

According to a preferred embodiment the rheology arrangement is provided as a belt for application to the subject of interest. A belt can easily be positioned at the subject of interest and cover its entire circumference.

In a further aspect of the present invention, the object is achieved by a magnetic resonance (MR) rheology imaging system, comprising a main magnet for generating a static magnetic field, a magnetic gradient coil system for generating gradient magnetic fields superimposed to the static magnetic field, an examination space provided to position a subject of interest within, at least one radio frequency (RF) antenna device for applying an RF field to the examination space to excite nuclei of the subject of interest, a control unit for controlling the at least one RF antenna device, and at least one rheology module as specified above. The control unit is connected to the at least one rheology module and adapted to control the at least one rheology module, so that the MR rheology imaging system can autonomously introduce oscillations into the subject of interest and perform the required MR measurements. Preferably, information from RF antenna units installed on the MR imaging system is combined with information received from the rheology module.

According to a preferred embodiment the magnetic resonance (MR) rheology imaging system comprises a rheology arrangement as specified above, whereby the rheology arrangement comprises the at least one rheology module. Preferably, information from RF antenna units installed on the MR imaging system are combined with information received from the rheology module(s) and the RF antenna module(s).

### BRIEF DESCRIPTION OF THE DRAWINGS

These and other aspects of the invention will be apparent from and elucidated with reference to the embodiments described hereinafter. Such an embodiment does not necessarily represent the full scope of the invention, however, and reference is made therefore to the claims and herein for interpreting the scope of the invention.

In the drawings:

FIG. 1 is a schematic illustration of a part of an embodiment of a magnetic resonance (MR) imaging system in accordance with the invention,

FIG. 2 is a perspective view of a rheology module according to a first embodiment in accordance with the invention,

FIG. 3 is a top view of the rheology module according to FIG. 2,

FIG. 4 is a perspective view of a rheology module according to a second embodiment in accordance with the invention,

FIG. 5 is a schematic illustration of a rheology module according to a third embodiment in accordance with the invention,

FIG. 6 is a schematic illustration of a rheology module according to a fourth embodiment in accordance with the invention,

FIG. 7 is a schematic illustration of a rheology module according to a fifth embodiment in accordance with the invention, and

FIG. 8 is a schematic illustration of a rheology arrangement comprising a rheology module in accordance with the invention.

### DETAILED DESCRIPTION OF EMBODIMENTS

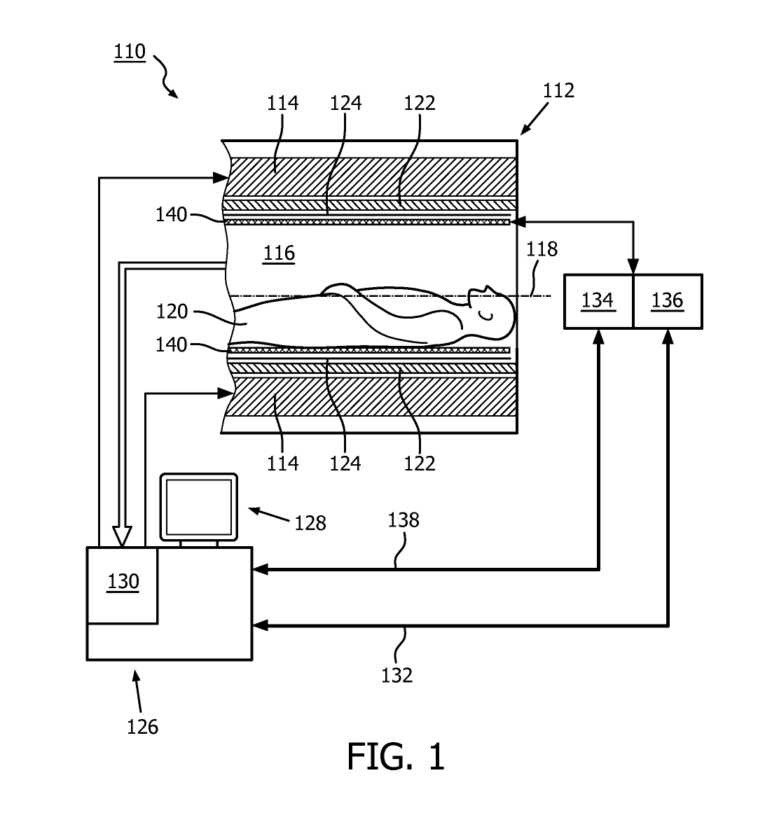

FIG. 1 shows a schematic illustration of a part of an embodiment of a magnetic resonance (MR) imaging system 110 comprising an MR scanner 112. The MR imaging system 110 includes a main magnet 114 provided for generating a static magnetic field. The main magnet 114 has a central bore that provides an examination space 116 around a center axis 118 for a subject of interest 120, usually a patient, to be positioned within. In this embodiment, the central bore and therefore the static magnetic field of the main magnet 114 has a horizontal orientation in accordance with the center axis 118. In an alternative embodiment, the orientation of the main magnet 114 can be different, e.g. to provide the static magnetic field with a vertical orientation. Further, the MR imaging system 110 comprises a magnetic gradient coil system 122 provided for generating gradient magnetic fields superimposed to the static magnetic field. The magnetic gradient coil system 122 is concentrically arranged within the bore of the main magnet 114, as known in the art.

Further, the MR imaging system 110 includes a radio frequency (RF) antenna device 140 designed as a whole-body coil having a tubular body. The RF antenna device 140 is provided for applying an RF magnetic field to the examination space 116 during RF transmit phases to excite nuclei of the subject of interest 120. The RF antenna device 140 is also provided to receive MR signal from the excited nuclei during RF receive phases. In a state of operation of the MR imaging system 110, RF transmit phases and RF receive phases are taking place in a consecutive manner. The RF antenna device 140 is arranged concentrically within the bore of the main magnet 114. As is known in the art, a cylindrical metal RF screen 124 is arranged concentrically between the magnetic gradient coil system 122 and the RF antenna device 140.

Moreover, the MR imaging system 110 comprises an MR image reconstruction unit 130 provided for reconstructing MR images from the acquired MR signals and an MR imaging system control unit 126 with a monitor unit 128 provided to control functions of the MR scanner 112, as is commonly known in the art. Control lines 132 are installed between the MR imaging system control unit 126 and an RF transmitter unit 134 that is provided to feed RF power of an MR radio frequency to the RF antenna device 140 via an RF switching unit 136 during the RF transmit phases. The RF switching unit 136 in turn is also controlled by the MR imaging system control unit 126, and another control line 138 is installed between the MR imaging system control unit 126 and the RF switching unit 136 to serve that purpose. During RF receive phase, the RF switching unit 136 directs the MR signals from the RF antenna device 140 to the MR image reconstruction unit 130 after pre-amplification.

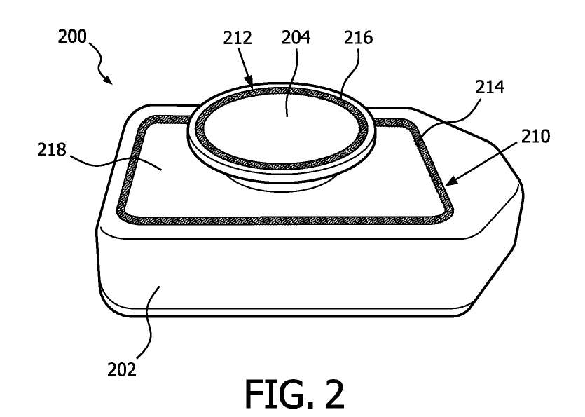

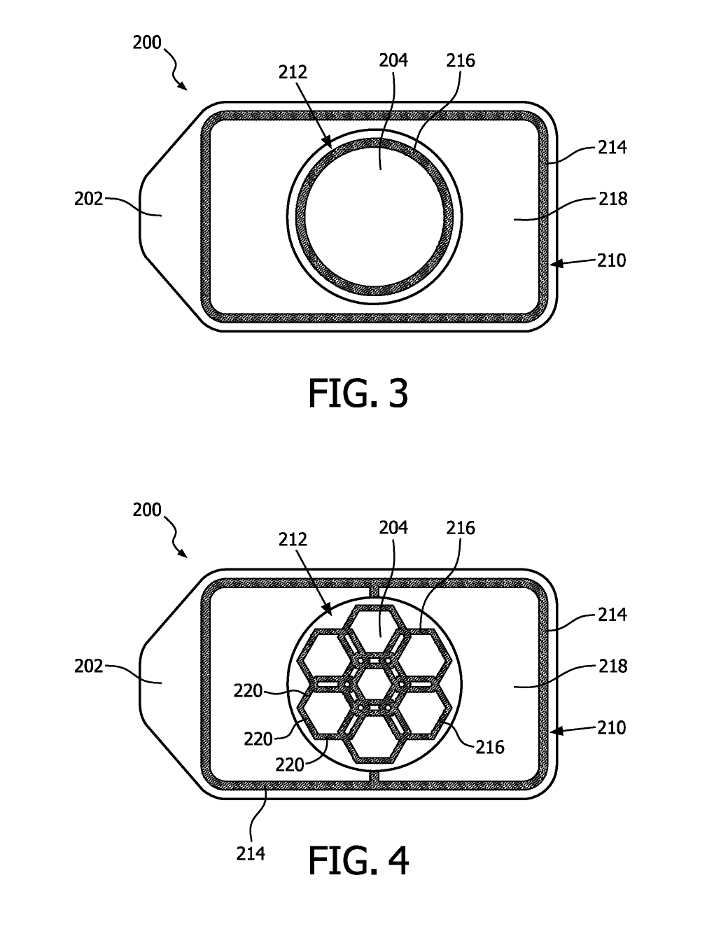

The MR imaging system 110 is provided as a MR rheology system comprising a rheology module 200, which is shown in FIGS. 2 and 3, and which is adapted to introduce mechanical oscillations into the subject of interest 120.

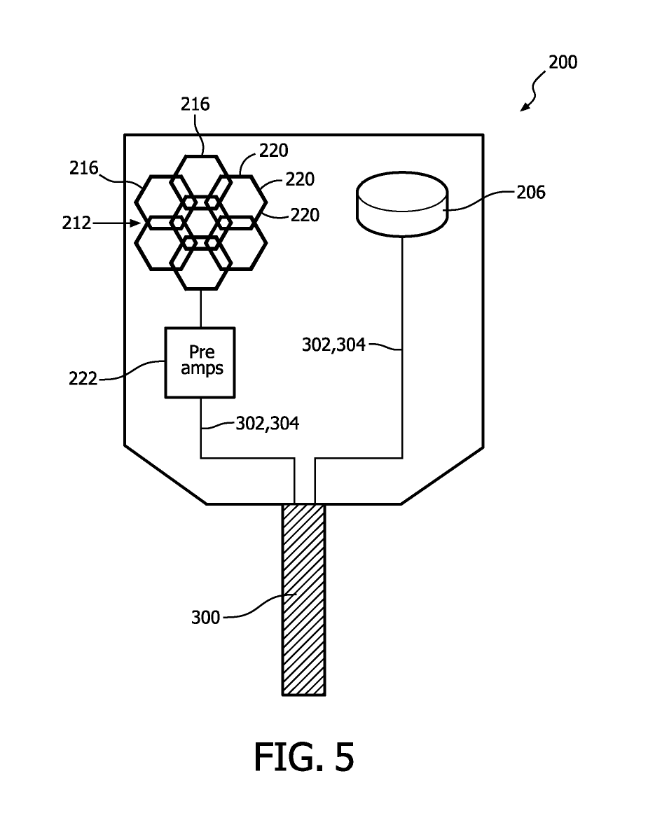

The rheology module 200 comprises a thin and flexible housing 202 and a mechanical oscillator unit 204, which is provided to be in contact with the subject of interest 120 in use. The oscillator unit 204 in this embodiment extends partially outside the housing 202 and is movable relative thereto. The rheology module 200 further comprises a transducer 206, which is indicated schematically in FIGS. 5 to 7, for moving the oscillator unit 204. The transducer 206 in this embodiment is an electromechanical transducer, which converts electric energy into mechanical energy, i.e. into mechanical oscillations. The rheology module 200 further comprises a local, heavy mass, which is not shown in the figures and which is MR compatible, to provide mechanical inertia to keep the amplitude of the mechanical oscillations high. The local mass is exchangeable with respect to a desired application. In an alternative embodiment, the mass can be omitted in case the subject of interest is placed in such a manner that a table, on which the subject of interest can be placed, counters the rheology unit. Also, the MR rheology unit can be fixed to the patient bed or an inner wall of a bore of the MR rheology imaging system.

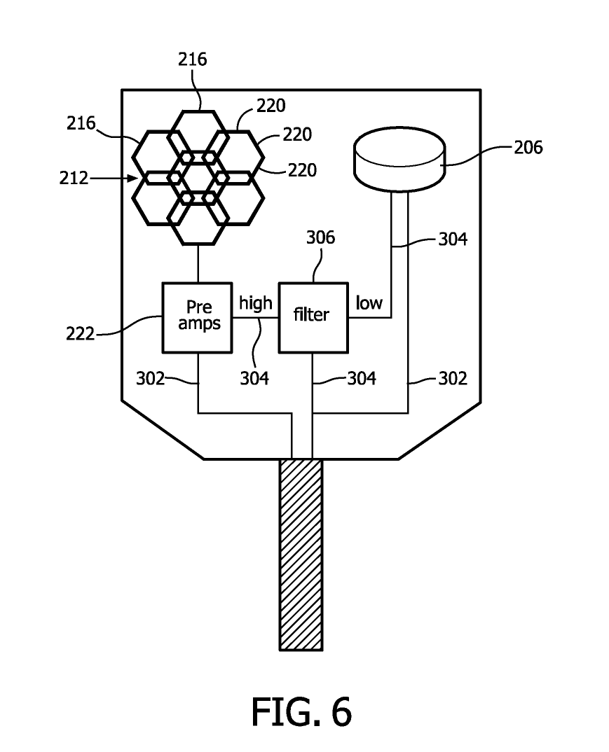

The rheology module 200 of this embodiment is provided with two RF antenna units 210, 212, which are respectively located at the housing 202 and the oscillator unit 204. Each RF antenna unit 210, 212 in this embodiment comprises one RF coil 214, 216, respectively. The RF coil 214 of the RF antenna unit 210 located at the housing 202 is provided at an upper face 218 of the housing 202, through which the oscillator unit 204 is connected to the transducer 206. The RF coil 214 is provided at an upper surface of the housing 202, i.e. at an upper surface of the upper face 218, and has a rectangular shape extending along the sides of the upper face 218. Accordingly, the RF coil 214 surrounds the oscillator unit 204. The RF coil 216 of the RF antenna unit 212 located at the oscillator unit 204 has a circular shape and is provided at an upper surface of the oscillator unit 204. The rheology module 200 also comprises pre-amplifiers 220, which are provided within the housing 202 for driving the RF coils 214, 216, as indicated in FIGS. 5 and 6.

The control unit 126 of the MR rheology imaging system 110 is connected to the rheology module 200 and adapted to control the rheology module 200, so that the MR rheology imaging system 110 can autonomously introduce mechanical oscillations into the subject of interest 120 and perform MR rheology imaging operations. A physical connection between the rheology module 200 and the control unit 126 is described in detail below.

A second embodiment of the rheology module 200 is shown in FIG. 4. The rheology module 200 is similar to the rheology module 200 of the first embodiment, so that only the differences will be described in detail.

The rheology module 200 of the second embodiment differs from that of the first embodiment in the structure of the RF antenna units 210, 212. According to the second embodiment, the RF antenna unit 210 located at the housing 202 comprises two rectangular RF coils 214, which are provided at an upper face 218 of the housing 202 as described above. Each RF coil 214 extends over half the area of the upper face 218. The RF antenna unit 212 located at the oscillator unit 204 comprises a set of seven individual RF coils 216, each of which has an essentially circular shape formed by six linear segments 220. The RF coils 216 of the RF antenna unit 216 are arranged in an array overlapping with adjacent RF coils 216. The rheology module 200 also comprises pre-amplifiers 222, which are provided within the housing 202 for driving the RF coils 214, 216, as indicated in FIGS. 5 and 6. Although the pre-amplifiers 222 are indicated in FIGS. 5 and 6 as a single box, each RF coil 214, 216 has one pre-amplifier 222 associated thereto.

The control unit 126 of the MR rheology imaging system 110 is connected to the rheology module 200 and adapted to control the rheology module 200, so that the MR rheology imaging system 110 can autonomously introduce oscillations into the subject of interest 120 and perform MR rheology imaging operations. The physical connection between the rheology module 200 and the control unit 126 is described in detail below.

FIG. 5 shows a rheology module 200 according to a third embodiment with a physical connection. By way of example, the physical connection is illustrated based on the rheology module 200 of the second embodiment, as indicated by the RF antenna device 212 located in the oscillator unit 204 having multiple RF coils 216. Nevertheless, the connection can be realized without general modifications for other rheology modules 200, e.g. that of the first embodiment.

The rheology module 200 according to the third embodiment comprises an electrical connector 300, which is provided as a single harness 300. The electrical connector 300 provides electrical power and a signaling connection to the transducer 206 and the RF coils 214, 216 of the RF antenna units 210, 212. The electrical connector 300 comprises an individual power line 302 and signaling lines 304 for the transducer 206 and the RF antenna units 210, 212. The power line 302 and the signaling lines 304 are indicated by a single line in FIG. 5. In particular, the pre-amplifiers 222 are connected by four lines 302, 304, which are DC feed as power line 302, as well as RF signal, detune, and malfunction detection as signaling lines 304. The transducer 206 is connected by two lines 302, 304, which are a driving signal and a sensing line to monitor the performance of the transducer 206. The signaling lines 304 are provided as bi-directional lines for sending signals to the transducer 206 and the RF antenna units 210, 212 and receiving signals from the transducer 206 and the RF antenna units 210, 212. The power lines 302 are B₀-compensated.

FIG. 6 shows a rheology module 200 according to a fourth embodiment with a physical connection. The rheology module 200 of the fourth embodiment only differs in the connection of its RF antenna units 210, 212 and transducer 206 to a harness 300 from the rheology module 200 of the third embodiment. Accordingly, only the differences between these rheology modules 200 will be discussed.

The rheology module 200 according to the fourth embodiment comprises an electrical connector 300, which is provided as a single harness 300. The harness 300 comprises power lines 304, which are directly connected to the transducer 206 and pre-amplifiers 222 of the RF antenna units 210, 212. The harness 300 further comprises a signaling line 304, which is connected to a filter unit 306 of the rheology module 200. The filter unit 306 is adapted for splitting signals received from the signaling line 304 according to their frequency.

Electrical signals on the signaling line 304 are provided from the filter unit 306 to the transducer 206 and the RF antenna units 210, 212 depending on their frequency. MR signals having a typical frequency of some 10 MHz are provided to the RF antenna units 210, 212, and a driving signal having some 10 Hz is provided to the transducer 206. A threshold for splitting the signals is defined between these frequencies.

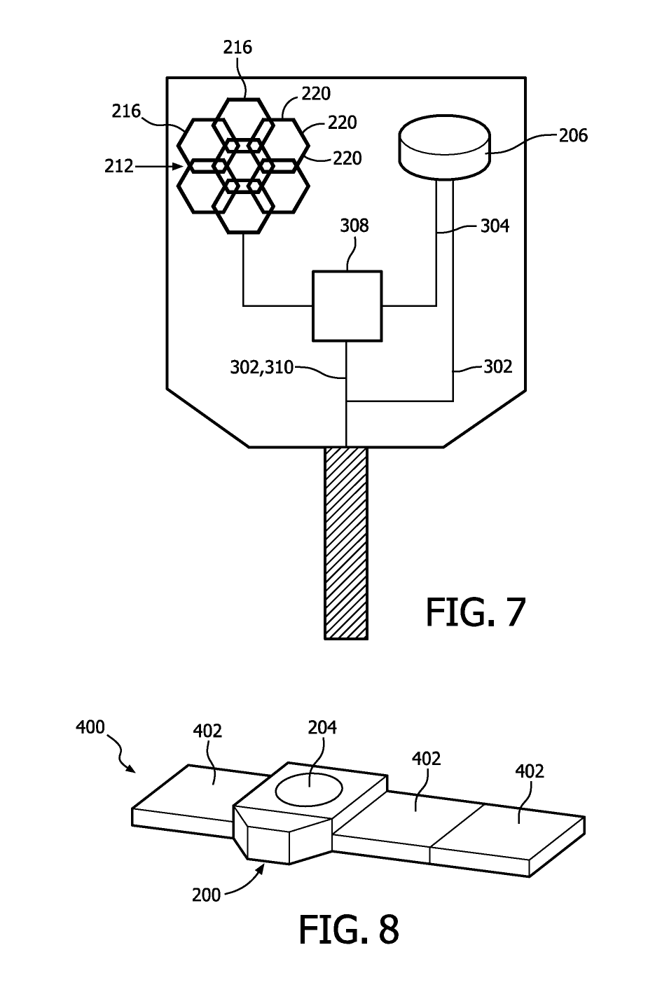

FIG. 7 shows a rheology module 200 according to a fifth embodiment with a physical connection. The rheology module 200 of the fifth embodiment only differs in the connection of its RF antenna units 210, 212 and transducer 206 to a harness 300 from the rheology module 200 of the third embodiment. Accordingly, only the differences between these rheology modules 200 will be discussed.

The rheology module 200 according to the fifth embodiment comprises an electrical connector 300, which is provided as a single harness. The rheology module 200 comprises an AD/DA converter unit 308, which is in this embodiment integrally provided with pre-amplifiers 222 in a driving box 308. The driving box 308 is connected to a digital signaling line 310 and a power line 302, whereby the power line 302 provides power for the pre-amplifiers 222. A transducer 206 is connected with a separate power line 302 of the harness 300. The signaling line 310 is used for signaling to/and from the transducer 206 and the preamplifiers 222 in the driving box 308. An analog signaling line 304 is provided between the transducer 206 and the AD/DA converter unit 308. The AD/DA converter unit 308 performs an AD/DA conversion. Additionally, an allocation of signals between the digital signaling line and the transducer 206 and the RF antenna unit, i.e. the pre-amplifiers 222 is performed, so that all signals are multiplexed on the digital signaling line 310. The digital signaling line 310 is a bi-directional line.

In an alternative embodiment the digital signaling line 310 in the harness 300 is an optical digital signaling line.

A sixth embodiment refers to a rheology arrangement 400 for use in a magnetic resonance (MR) rheology imaging system 110, which is shown in FIG. 8. The rheology arrangement 400 comprises three RF antenna modules 402, which comprises a RF antenna unit as described above in respect to the rheology modules 200, and one rheology module 200 as specified above. The modules 200, 402 are interconnected and arranged in a chain, so that the rheology arrangement 400 can be used as a belt for application to the subject of interest 120. The modules 200, 402 are attached to each other by Velcro fasteners, which are not explicitly shown in FIG. 8. In an alternative embodiment, the modules 200, 402 comprise electrical connectors for connecting to adjacent modules 200, 402, and the rheology arrangement 400 comprises a single connector for electrically connecting all modules 200, 402 to the MR rheology imaging system 110.

According to a modified embodiment, the magnetic resonance (MR) rheology imaging system 110 comprises a rheology arrangement 400, whereby the rheology arrangement 400 comprises the at least one rheology module 200.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

110 magnetic resonance (MR) imaging system
112 magnetic resonance (MR) scanner
114 main magnet
116 RF examination space
118 center axis
120 subject of interest
122 magnetic gradient coil system
124 RF screen
126 MR imaging system control unit
128 monitor unit
130 MR image reconstruction unit
132 control line
134 RF transmitter unit
136 RF switching unit
138 control line
140 radio frequency (RF) antenna device
200 rheology module
202 housing
204 oscillator unit
206 transducer
210 RF antenna unit (at housing)
212 RF antenna unit (at oscillator)
214 RF coil (at housing)
216 RF coil (at oscillator)
218 upper face
220 linear segment
222 pre-amplifier
300 electrical connector, harness
302 power line
304 signaling line
306 filter unit
308 AD/DA converter unit
310 digital signaling line
400 rheology arrangement
402 RF antenna module

1. A rheology module for use in a magnetic resonance (MR) rheology imaging system, whereby the rheology module is adapted to introduce mechanical oscillations into a subject of interest, comprising
a housing,
a mechanical oscillator unit, which is arranged at least partially inside the housing, extends at least partially outside the housing and is movable as a whole relative to the housing, and
a transducer for moving the oscillator unit,
whereby
the rheology module comprises at least one radio frequency (RF) antenna unit, which comprises at least one RF coil.

2. The rheology module according to claim 1, whereby
at least one RF antenna unit is located on or in the housing.

3. The rheology module according to claim 1, whereby
at least one RF antenna unit is located on or in the oscillator unit.

4. The rheology module according to claim 1, whereby
at least one RF antenna unit comprises a set of multiple RF coils.

5. The rheology module according to claim 1, whereby
the transducer converts electrical energy into mechanical oscillations, and
the rheology module comprises an electrical connector,
whereby
the electrical connector is provided as a single harness to provide electrical power and a signaling connection to the transducer and the at least one RF antenna unit.

6. The rheology module according to claim 5, whereby
the harness comprises at least one electrical line, which is connected to the transducer and the at least one RF antenna unit, and
a filter unit is provided in the electrical line for splitting signals received from the electrical line according to their frequency,
whereby
electrical signals on the electrical line are provided from the filter unit to the transducer and the RF antenna unit depending on their frequency.

7. The rheology module according to claim 5, whereby
the harness comprises at least one digital signaling line,
the rheology module comprises an AD/DA converter unit which is connected to the digital signaling line, the transducer and the at least one RF antenna unit, and
the AD/DA converter unit is adapted to perform a conversion and allocation of signals between the digital signaling line and the transducer and the at least one RF antenna unit.

8. The rheology module according to claim 7, whereby
the digital signaling line is an optical signaling line.

9. The rheology module according to claim 7, whereby
the housing is flexible.

10. A rheology arrangement for use in a magnetic resonance (MR) rheology imaging system, comprising
at least one RF antenna module comprising at least one RF antenna unit, and
at least one rheology module according to claim 1,
whereby
the at least one RF antenna module and the at least one rheology module are interconnected.

11. The rheology arrangement according to claim 10, whereby
the rheology arrangement is provided as a belt for application to the subject of interest.

12. A magnetic resonance (MR) rheology imaging system, comprising
a main magnet for generating a static magnetic field,
a magnetic gradient coil system for generating gradient magnetic fields superimposed to the static magnetic field,
an examination space provided to position a subject of interest within,
at least one radio frequency (RF) antenna device for applying an RF field to the examination space to excite nuclei of the subject of interest,
a control unit for controlling the at least one RF antenna device, and
at least one rheology module according to claim 1.

13. The magnetic resonance (MR) rheology imaging system according to claim 12, comprising
a rheology arrangement, whereby
the rheology arrangement comprises the at least one rheology module.
